(19) United States
(12) Patent Application Publication
(10) Pub. No.: US 2010/0036139 A1
(43) Pub. Date: Feb. 11, 2010

(54) PROCESS FOR THE PREPARATION OF BENZOFURAN-2-CARBOXAMIDES

(75) Inventor: Andreas Bathe, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, Darmstadt (DE)

(21) Appl. No.: 12/519,885

(22) PCT Filed: Nov. 22, 2007
(86) PCT No.: PCT/EP2007/010123

§ 371 Date: Jun. 18, 2009

(30) Foreign Application Priority Data
Dec. 21, 2006 (DE) 10 2006 060 597.7

(51) Int. Cl. C07D 307/80

(52) U.S. Cl. 549/468

(57) ABSTRACT

The invention relates to a process for the preparation of benzofuran-2-carboxamides of the formula (I), in which R¹, R², R³, R⁴, R⁵ and R⁶ have the meanings indicated in Claim 1.

The invention relates to a process for the preparation of benzofuran-2-carboxamides of the formula I

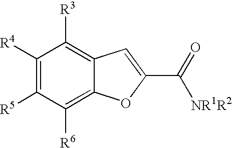

in which
R¹, R² denote H or cycloalkyl having 3 to 7 C atoms or unbranched or branched alkyl having 1 to 10 C atoms, each of which is unsubstituted or substituted by A, where one or more CH2 groups of the alkyl group may be replaced by an O or S atom, by CH═CH groups or by C═C groups or in which one or more hydrogen atoms of the alkyl group may be replaced by Hal, OH, Ar, Het, cycloalkyl having 3 to 10 C atoms, N(R⁷)₂ CN, COOR⁷, CON(R⁷)₂, NR⁷COR⁷, NR⁷CON(R⁷)₂ NR⁷SO₂A or SO₂NR⁷
NR¹R² together denotes a three- to 7-membered saturated heterocyclic ring, in which, in addition, 1 or 2 N and/or 1 or 2 S and/or 1 or 2 O atoms and/or one S(O)ₘ group may be present, which may be substituted by A, Hal, cycloalkyl having 3 to 10 C atoms, OR⁷, N(R⁷)₂, CN, COOR⁷, CON(R⁷)₂NR⁷COR⁷ and/or carbonyl oxygen,
A denotes unbranched or branched alkyl having 1 to 6 C atoms, in which at least one CH₂ group may be replaced by an O or S atom, or by a CH═CH group, or at least one H atom may be replaced by F,
Ar denotes phenyl, naphthyl or biphenyl, each of which is unsubstituted or mono- or polysubstituted by Hal, A, OR⁷, N(R⁷)₂, NO₂, CN, COOR⁷, CON(R⁷)₂, NR⁷COR⁷, NR⁷CON(R⁷)₂, NR⁷SO₂A, COR⁷,

SO₂NR⁷ or S(O)ₘA,
Het denotes a saturated, unsaturated or aromatic mono- or bicyclic heterocyclic radical having 5 to 10 ring members, in which 1 to 4 N and/or 1 to 4 S and/or 1 to 4 O atoms may be present and the heterocyclic radical may be mono-, di- or trisubstituted by Hal, A, —[C(R⁷)₂]_{O}—Ar, —[C(R⁷)₂]_{O}-cycloalkyl, OR⁷, N(R⁷)₂, NO₂, CN, COOR⁷, CON(R⁷)₂, NR⁷COA, NR⁷CON(R⁷)₂, NR⁷SO₂A, COR⁷, SO₂NR⁷ or S(O)ₘA and/or carbonyl oxygen,
Hal denotes F, Cl, Br or I,
n denotes 2, 3, 4 or 5,
m denotes 1 or 2,
o denotes 1, 2, 3 or 4,
R³, R⁴, R⁵, each, independently of one another, denote H, A or alkoxy having
R⁶ 1-20 C atoms, Ar, aryloxy or COOR₇, Hal, OH, CN, NO₂, N(R⁷)₂, NHCOR⁷, CH₂OH, CH₂OR⁷ or CO(R⁷)₂, and one of the radicals R³, R⁴, R⁵, R⁶, in particular the radical R⁴, instead also denotes 4-benzylpiperazinyl, 4-tert-butoxycarbonylpiperazin-1-yl, a leaving group or

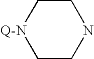

R⁷ denotes H or A
Q denotes an amino-protecting group,
by reaction of compounds of the formula II and III in the presence of a suitable base

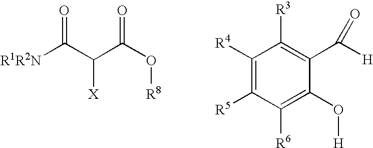

in which
X denotes Hal,
R⁸ denotes A and
R¹-R⁷ have the meaning indicated above.

The invention facilitates, in particular, the synthesis of substituted benzofuran-2-carboxamides which are suitable as starting compounds in the preparation of medicaments, such as, for example, antidepressants. In particular, it opens up the possibility of preparing 5-(4-tert-butoxycarbonyl-1-piperazinyl)benzofuran-2-carboxamide in a simple manner. Compounds of this type are precursors for the synthesis of antidepressants, for example for the antidepressant EMD 68843 (vilazodone)

To date, two processes have been described for the preparation of (I) from substituted salicylaldehydes. In these, substituted salicylaldehydes were reacted either with XCH₂COOR or with XCH(COOR)₂, where R stands for an alkyl radical. An amidation was subsequently carried out. The processes known to date are therefore multicut processes with relatively low yields.

The invention was therefore based on the object of developing a process for the preparation of compounds of the formula (I) or salts thereof which both represents a simplification (one-step process) and has a higher yield.

It has been found that the compounds of the formula I and salts thereof, which are important intermediates for the preparation of medicaments—in particular of those which act, for example, on the central nervous system—can be obtained by reaction of compounds of the formula II or salts thereof with compounds of the formula III or salts thereof.

Above and below, the radicals R¹ to R⁸, as well as Q and X have the meanings indicated for the formulae I to III, unless expressly indicated otherwise.

The radical A denotes unbranched or branched alkyl and has 1 to 6, preferably 1, 2, 3 or 4, in particular 1 or 2, C atoms. Alkyl therefore denotes, in particular, for example, methyl, furthermore ethyl, n-propyl, isopropyl, n-butyl, sec-butyl or tert-butyl, furthermore also pentyl, 1-, 2- or 3-methylbutyl, 1,1-, 1,2- or 2,2-di-methylpropyl, 1-ethylpropyl, hexyl, 1-, 2-, 3- or4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, in which it is possible for a CH₂ group to be replaced by an O or S atom or by a CH═CH group or for at least one H atom to be replaced by F. The radical A therefore furthermore denotes, for example, trifluoromethyl, pentafluoroethyl, heptafluoropropyl, methoxymethyl, methoxyethyl, methoxypropyl, ethoxymethyl, ethoxyethyl, ethoxypropyl, methylsulfanylmethyl, methylsulfanylethyl, methylsulfanylpropyl, ethylsulfanylmethyl, ethylsulfanylethyl, ethylsulfanylpropyl, allyl, propenyl, but-2-enyl, but-3-enyl, pent-3-enyl, pent-4-enyl or hex-3-enyl.

Aryl or Ar denotes phenyl, naphthyl or biphenyl, each of which is unsubstituted or mono- or polysubstituted by Hal, A, OR⁷, N(R⁷)₂, NO₂, CN, COOR⁷, CON(R⁷)₂, NR⁷COR⁷, NR⁷CON(R⁷)₂, NR⁷SO₂A, SO₂NR⁷ or S(O)ₘA, where A has one of the meanings indicated above and R⁷ and m have one of the meanings indicated below.

Ar is preferably unsubstituted or substituted phenyl, naphthyl or biphenyl, specifically preferably phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-trifluoromethylphenyl, o-, m- or p-hydroxyphenyl, o-, m- or p-nitrophenyl, o-, m- or p-(trifluoromethoxy)phenyl, o-, m- or p-cyanophenyl, o-, m- or p-methoxyphenyl, o-, m- or p-ethoxyphenyl, o-, m- or p-fluorophenyl, o-, m- or p-bromophenyl, o-, m- or p- chlorophenyl, o-, m- or p-(difluoromethoxy)phenyl, o-, m- or p-(fluoromethoxy)phenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2-chloro-3-methyl-, 2-chloro-4-methyl-, 2-chloro-5-methyl-, 2-chloro-6-methyl-, 2-methyl-3-chloro-, 2-methyl-4-chloro-, 2-methyl-5-chloro-, 2-methyl-6-chloro-, 3-chloro-4-methyl-, 3-chloro-5-methyl- or 3-methyl-4-chlorophenyl, 2-bromo-3-methyl-, 2-bromo-4-methyl-, 2-bromo-5-methyl-, 2-bromo-6-methyl-, 2-methyl-3-bromo-, 2-methyl-4-bromo-, 2-methyl-5-bromo-, 2-methyl-6-bromo-, 3-bromo-4-methyl-, 3-bromo-5-methyl- or 3-methyl-4-bromophenyl, 2,4- or 2,5-dinitrophenyl, 2,5- or 3,4-dimethoxyphenyl, 3-nitro-4-chlorophenyl, 2,3,4-, 2,3,5-, 2,3,6-, 2,4,6- or 3,4,5-trichlorophenyl, 2,4,6-tri-tert-butylphenyl, furthermore preferably 2-nitro-4-(trifluoromethyl)phenyl, 3,5-di-(trifluoromethyl)phenyl, 2,5-dimethylphenyl, 2-hydroxy-3,5-dichlorophenyl, 2-fluoro-5- or 4-fluoro-3-(trifluoromethyl)phenyl, 4-chloro-2- or 4-chloro-3-(trifluoromethyl)-, 2-chloro-4- or 2-chloro-5-(trifluoromethyl)phenyl, 4-bromo-2- or 4-bromo-3-(trifluoromethyl)phenyl, p-iodophenyl, 2-nitro-4-methoxyphenyl, 2,5-dimethoxy-4-nitrophenyl, 2-methyl-5-nitrophenyl, 2,4-dimethyl-3-nitrophenyl, 4-fluoro-3-chlorophenyl, 4-fluoro-3,5-dimethylphenyl, 2-fluoro-4-bromophenyl, 2,5-difluoro-4-bromophenyl, 2,4-dichloro-5-methylphenyl, 3-bromo-6-methoxyphenyl, 3-chloro-6-methoxyphenyl, 2-methoxy-5-methylphenyl or 2,4,6-triisopropylphenyl. Ar is particularly preferably phenyl.

Cycloalkyl having 3 to 10 C atoms which is unsubstituted or substituted by A preferably denotes cyclopropyl, cyclobutyl, cyclopentyl, cyclohexyl, 4-methylcyclohexyl, cycloheptyl or cyclooctyl. Cycloalkyl likewise denotes mono- or bicyclic terpenes, preferably p-menthane, menthol, pinane, bornane or camphor, where each known stereoisomeric form is included, or adamantyl. For camphor, this means both L-camphor and also D-camphor. Cycloalkyl is particularly preferably cyclopropyl, cyclobutyl, cyclopentyl, cyclohexyl, cycloheptyl and 4-methylcyclohexyl.

Hal denotes fluorine, chlorine, bromine or iodine, in particular chlorine or bromine. In compounds of the formula 1, Hal particularly preferably denotes fluorine.

Het denotes a saturated, unsaturated or aromatic mono- or bicyclic heterocyclic radical having 5 to 10 ring members, in which 1 to 4 N and/or 1 to 4 S and/or 1 to 4 O atoms may be present and the heterocyclic radical may be mono-, di- or trisubstituted by Hal. A, —[C(R⁷)₂]_{O}—Ar, —[C(R⁷)₂]ₒ-cycloalkyl. OR⁷, N(R⁷)₂, NO₂, CN, COOR⁷, CON(R⁷)₂, NR⁷COA, NR⁷CON(R⁷)₂, NR⁷SO₂A, COR⁷, SO₂NR⁷ or S(O)ₘA and/or carbonyl oxygen, where A and cycloalkyl have one of the meanings indicated above and R⁷, m and o have one of the meanings indicated below.

Het is preferably substituted or unsubstituted 2- or 3-furyl, 2- or 3-thienyl, 1-,2- or 3-pyrrolyl, 1-, 2-, 4- or 5-imidazolyl, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-l-1-, -4- or -5-yl, 1,2,4-triazol-1-, -4 or -5-yl, 1- or 5-tetrazolyl, 1,2,3-oxadiazol-4- or -5-yl 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 4-4H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, l-, 2-, 3-, 4-, 5-, 6- or 7-1H-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 4- or 5-benzothiadiazolyl, 2-, 4-, 5-, 6- or 7-benzisothiazolyl, 4-, 5-, 6- or 7-benz-2,1,3-oxadiazolyl, 1-, 2-, 3-, 4-, 5-, 6-, 7- or 8-quinolinyl, 1-, 3-, 4-, 5-, 6-, 7- or 8-isoquinolinyl, 1-, 2-, 3-, 4- or 9-carbazolyl, 1-, 2-, 3-, 4-, 5-, 6-, 7-, 8- or 9-acridinyl, 3-, 4-, 5-, 6-, 7- or 8- cinnolinyl, 2-, 4-, 5-, 6-, 7- or 8-quinazolinyl. The heterocyclic radicals may also be partially or fully hydrogenated. Het may thus also denote 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -3-pyrollyl,tetrahydro-1-, -2- or 4-imidazolyl, 2,3-dihydro-1-, -2-, -3-, -4-, -5-, -6-, -7-1 H-indolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1,2,3,6-tetrahydro-1-, -2-, -3,-4-, -5- or -6-pyridyl, 1-, 2-, 3- or 4-piperidinyl, 1-, 2-, 3- or 4azepanyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolinyl, 1,2,3,4-tetrahydro-1-, -2-, -3-, -4-, -5-, -6-, -7- or -8-isoquinol

Het is particularly preferably unsubstituted furan-2-yl, tetrahydrofuran-2-yl, pyridin-4-yl, pyridin-3-yl, pyridin-2-yl, thiophen-2-yl or imidazol-5-yl.

R¹ denotes cycloalkyl having 3 to 10 C atoms or unbranched or branched alkyl having 1 to 10 C atoms, each of which is unsubstituted or substituted by A and in which one or more CH₂ groups of the alkyl group may be replaced by an O or S atom, by CH═CH groups or by C═C groups or in which one or more hydrogen atoms of the alkyl group may be replaced by Hal, OH, Ar, Het, cycloalkyl having 3 to 10 C atoms, N(R⁷)₂, CN, COOR⁷, CON(R⁷)₂, NR⁷COR⁷, NR⁷CON(R⁷)₂, NR⁷SO₂A or SO₂NR⁷, where A, Ar, Hal, Het and cycloalkyl have one of the meanings indicated above and R⁷ has one of the meanings indicated below.

R¹ furthermore preferably denotes H, allyl, benzyl, phenylethyl, 2-methoxyethyl, 3-methoxypropyl, 3-ethoxypropyl, aminocarbonylmethyl, 2-dimethylaminoethyl, 2-diethylaminoethyl, 3-dimethylaminopropyl, 4-dimethylaminobutyl, 2-methylaminoethyl, cyanomethyl, 2,2,2-trifluoroethyl, 2-hydroxyethyl, 3-hydroxypropyl, prop-2-ynyl, cyclopropyl, cyclobutyl, cyclopentyl, cyclohexyl, cycloheptyl, 4-methylcyclohexyl, cyclohexylmethyl, furan-2-ylmethyl, 2-morpholin-4-ylethyl, pyridin-3-ylmethyl, pyridin-2-ylmethyl, pyridin-4-ylmethyl, 2-imidazol-5-ylethyl, thiophen-2-ylmethyl or tetrahydrofuran-2-ylmethyl.

R² denotes H, A or R¹, where A and R¹ have one of the meanings indicated above. R² is particularly preferably H.

NR¹R² together denotes a three- to 7-membered saturated heterocyclic ring, in which, in addition, 1 or 2 N and/or 1 or 2 S and/or 1 or 2 O atoms and/or one S(O)ₘ group may be present, which may be substituted by A, Hal, cycloalkyl having 3 to 10 C atoms, OR⁷, N(R⁷)₂, CN, COOR⁷, CON(R⁷)₂, NR⁷COR⁷ and/or carbonyl oxygen, where A, Hal and cycloalkyl have one of the meanings indicated above and R⁷ and m have one of the meanings mentioned below.

NR¹R² is preferably 1-, 2- or 3-pyrrolidinyl, tetrahydro-1-, -2- or 4-imidazolyl, tetrahydro-1-, -3- or -4-pyrazolyl, 1-, 2,-3- or 4-piperidinyl, 1-, 2,-, 3- or 4-azepanyl, 2-, 3- or 4-morpholinyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl or 1-, 2- or 3- piperazinyl. NR¹R² is particularly preferably piperidin-1-yl and morpholin-1-yl.

n denotes 2, 3, 4 or 5, particularly preferably 4.

m denotes 1 or 2.

o denotes 0, 1, 2, 3 or 4.

Leaving group denotes a group which leaves during the reaction. Particularly suitable for this purpose are the groups Hal, such as Cl or Br, toluenesulfonyl, benzenesulfonyl, methanesulfonyl, and trifluoromethanesulfonyl.

The term amino-protecting group relates to groups which are capable of protecting amino groups against chemical reactions, but can easily be removed after completion of the desired reaction—elsewhere in the molecule. Typical amino-protecting groups are, in particular, unsubstituted acyl, aryl, aralkoxymethyl or aralkyl groups.

Since the amino-protecting groups are removed after the desired reaction, type and size play a minor role. Nevertheless, amino-protecting groups having 1 to 20, preferably 1 to 8, C atoms are particularly preferred. Acyl groups are taken to mean derivatives of aliphatic, araliphatic, aromatic or heterocyclic carboxylic acids or sulfonic acids and in particular alkoxycarbonyl, aryloxycarbonyl and very particularly aralkoxycarbonyl groups. Examples of acyl groups of this type are alkanoyl, such as acetyl, propionyl, butyryl; aralkanoyl, such as phenylacetyl; aroyl, such as benzoyl or tolyl; aryloxyalkanoyl, such as phenoxyacetyl; alkoxycarbonyl, such as methoxycarbonyl, ethoxycarbonyl, 2,2,2-trichloroethoxycarbonyl, BOC (tert-butoxycarbonyl), 2-iodoethoxycarbonyl; aralkyloxycarbonyl, such as CBZ (carbobenzoxycarbonyl), 4-methoxybenzyloxycarbonyl, FMOC (9-fluorenylmethoxycarbonyl); arylsulfonyl, such as Mtr (4-methoxy-2,3,6-trimethylphenylsulfonyl).

In particular, amino-protecting group denotes benzyl or BOC.

R³, R⁴, R⁵ and R⁶ preferably denote, independently of one another, H, methyl or ethyl, but in particular H.

R⁴ preferably denotes, in particular, methyl, ethyl, 4-benzylpiperazinyl or 4-tert-butoxycarbonylpiperazin-1-yl or a leaving group, but in particular H or 4-tert-butoxycarbonylpiperazin-1-yl or a leaving group.

R⁷ and R⁸ preferably denote, independently of one another, methyl or ethyl, but in particular ethyl.

A particularly preferred compound of the formula II is bromomalonic acid monoethyl ester monoamide.

Particularly preferred compounds of the formula III are 5-(4-tert-butoxycarbonylpiperazin-1-yl)-2-hydroxybenzaldehyde or

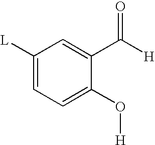

In which L denotes a leaving group and preferably Br. In the process according to the invention, the compound of the formula III is preferably dissolved or suspended in a solvent, such as, for example, water, alcohol,/ether, saturated or aromatic halogenated or halogen-free hydrocarbons or mixtures thereof, but in particular in polar aprotic solvents, such as, for example, dimethylformamide. A suitable base, such as, for example, potassium carbonate or alkali metal alkoxide, is added. The compound of the formula II is subsequently added and stirred for 1 to 12 h, preferably 1.5 to 8 h. The reaction mixture is warmed at 20° C. to 200° C., preferably at 50° C. to 180° C. and in particular at 80° C. to 150° C., for a further 1 to 24 h, preferably for 2 to 10 h. The target compound formed in this way is obtained by conventional work-up.

Conventional work-up preferably means filtration, addition of water and re-filtration.

Preferred embodiments of the process according to the invention are given below:

Process for the preparation of compounds of the formula I, characterised in that the reaction of the compounds of the formulae II with the compounds of the formula III to give compounds of the formula I is carried out at 0° C.-200° C.

Process for the preparation of compounds of the formula I, characterised in that the reaction of the compounds of the formulae II with the compounds of the formula III to give compounds of the formula I is carried out at pH 7 to 14, preferably at pH 8-11.

The substances prepared with the aid of the process according to the invention can serve as precursors for the synthesis of antidepressants, in particular for the antidepressant EMD 68843 (vilazodone).

° C. below denotes degrees Celsius.

### EXAMPLE 1

1.4 g of 2-hydroxybenzaldehyde are dissolved in 15 ml of dimethylformamide at 20° C. with stirring. 2.1 g of potassium carbonate are added and stirred for 15 min. 2.8 g of 2-bromomalonic acid monoethyl ester monoamide are subsequently metered in. The mixture is stirred at room temperature for 2 hrs., then stirred at 120° C. for 5 hrs. After cooling to 20° C., the solid component is filtered off; the filter residue is washed with 20 ml of dimethylformamide and discarded. The combined filtrates are slowly diluted with 150 ml of water with stirring and stirred at 20° C. for 1 hr. The solid product formed is filtered off, the filter residue is washed with water and dried. The solid formed is recrystallised from toluene/|n-heptane.

### EXAMPLE 2

3.5 g of 5-(4-tert-butoxycarbonylpiperazin-l-yl)-2-hydroxybenzaldehyde are dissolved in 15 ml of dimethylformamide at 20° C. with stirring. 2.1 g of potassium carbonate are added and stirred for 15 min. 2.8 g of 2-bromomalonic acid monoethyl ester monoamide are subsequently metered in. The mixture is stirred at room temperature for 2 hrs., then stirred at 120° C. for 5 hrs. After cooling to 20° C., the solid component is filtered off; the filter residue is washed with 20 ml of dimethylformamide and discarded. The combined filtrates are slowly diluted with 150 ml of water with stirring and stirred at 20° C. for 1 hr. The solid product formed is filtered off, the filter residue is washed with water and dried. The solid formed is recrystallised from tolueneln-heptane.

1. Process for the preparation of benzofuran-2-carboxamides of the formula I 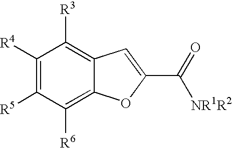
in which
R¹, R² denote H or cycloalkyl having 3 to 7 C atoms or unbranched or branched alkyl having 1 to 10 C atoms, each of which is unsubstituted or substituted by A, where one or more CH2 groups of the alkyl group may be replaced by an O or S atom, by CH═CH groups or by C═C groups or in which one or more hydrogen atoms of the alkyl group may be replaced by Hal, OH, Ar, Het, cycloalkyl having 3 to 10 C atoms, N(R⁷)₂ CN, COOR⁷, CON(R⁷)₂, NR⁷COR⁷, NR⁷CON(R⁷)₂, NR⁷SO₂A or SO₂NR⁷
NR¹R² together denotes a three- to 7-membered saturated heterocyclic ring, in which, in addition, 1 or 2 N and/or 1 or 2 S and/or 1 or 2 O atoms and/or one S(O)ₘ group may be present, which may be substituted by A, Hal, cycloalkyl having 3 to 10 C atoms, OR⁷, N(R⁷)₂, CN, COOR⁷, CON(R⁷)₂, NR⁷COR⁷ and/or carbonyl oxygen,
A denotes unbranched or branched alkyl having 1 to 6 C atoms, in which at least one CH₂ group may be replaced by an O or S atom, or by a CH═CH group, or at least one H atom may be replaced by F,
Ar denotes phenyl, naphthyl or biphenyl, each of which is unsubstituted or mono- or polysubstituted by Hal, A, OR⁷, N(R⁷)₂, NO₂, CN, COOR⁷, CON(R⁷)₂, NR⁷COR⁷, NR⁷CON(R⁷)₂, NR⁷SO₂A, COR⁷, SO₂NR⁷ or S(O)mA,
Het denotes a saturated, unsaturated or aromatic mono- or bicyclic heterocyclic radical having 5 to 10 ring members, in which 1 to 4 N and/or 1 to 4 S and/or 1 to 4 O atoms may be present and the heterocyclic radical may be mono-, di- or trisubstituted by Hal, A, —[C(R⁷)₂]_{O}—Ar, —[C(R⁷)₂]_{O}-cycloalkyl, OR⁷, N(R⁷)₂, NO₂, CN, COOR⁷, CON(R⁷)₂, NR⁷COA, NR⁷CON(R7)₂, NR⁷SO₂A, COR⁷, SO₂NR⁷ or S(O)ₘA and/or carbonyl oxygen,
Hal denotes F, CI, Br or I,
n denotes 2, 3, 4 or 5,
m denotes 1 or 2,
o denotes 0, 1, 2, 3 or 4,
R³, R⁴, R⁵, each, independently of one another, denote H, A or alkoxy having
R⁶ 1-20 C atoms, Ar, aryloxy or COOR₇, Hal, OH, CN, NO₂, N(R⁷)₂, NHCOR⁷, CH₂OH, CH₂OR⁷ or CO(R⁷)₂, and one of the radicals R³, R⁴, R⁵, R⁶, instead also denotes 4-benzylpiperazinyl, 4-tert-butoxy-carbonylpiperazin-1-yl, a leaving group or 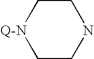
R⁷ denotes H orA
H or an amino-protecting group,
by reaction of compounds of the formula II and III in the presence of a suitable base 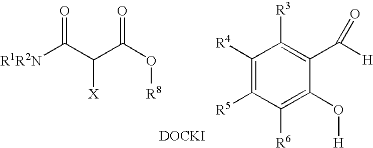
in which
x denotes Hal,
R⁸ denotes A and
R¹-R⁷ have the meaning indicated above

2. Process according to claim 1, in which R′ and R* simultaneously denote H.

3. Process according to claim 1, in which R⁴ denotes a leaving group, 4-tert-butoxycarbonyl-piperazin-1-yl or 4-benzylpiperazinyl.

4. Process according to claim 1, in which R³, R⁵ and R⁶, independently of one another, denote H or methyl.

5. Process according to claim 1, in which R⁷ denotes H.

6. Process according to claim 1, in which R⁸ denotes alkyl.

7. Process for the preparation of 5-(4-tert-butoxycarbonyl-1-piperazinyl)benzofuran-2-carboxamide according to claim 1.

8. Process according to claim 1, characterised in that the reaction of the comp. of the formula II with the comp. of the formula III is carried out in a polar aprotic solvent.

9. Process for the preparation of compounds of the formula I according to claim 1, characterised in that the reaction of the compounds of the formulae II with the compounds of the formula III to give compounds of the formula I is carried out at pH values between 7 and 14 and at temperatures of 0-200° C.

10. Process according to claim 1, characterised in that the compound of the formula III employed is 5-(4-tert-butoxycarbonylpiperazin-1-yl)-2-hydroxybenzaldehyde.
